(19) United States
(12) Patent Application Publication
(10) Pub. No.: US 2013/0172808 A1
(43) Pub. Date: Jul. 4, 2013

(54) MEDICAL INFUSION DEVICE PRODUCING ADENOSINE TRIPHOSPHATE FROM CARBOHYDRATES

(71) Applicant: G. Ford Gilbert, McClellan, CA (US)

(72) Inventor: G. Ford Gilbert, McClellan, CA (US)

(21) Appl. No.: 13/341,813

(22) Filed: Dec. 30, 2011

(51) Int. Cl. A61M 5/168

(52) U.S. Cl. 604/66

(57) ABSTRACT

A medical infusion system which increases adenosine triphosphate from carbohydrates by a pump delivering precisely timed and calculated boluses of hormones such as insulin resulting in oscillations of hormones in whole blood sufficient to cover a carbohydrate load equal to no less than forty percent (40%) of the minimum daily allowance for carbohydrates where the blood glucose is from 60 mg/dl to 300 mg/dl. The system comprises a pump and optimally a cassette or cartridge where the plunger rotates as it advances in reference to the cartridge to provide additional accuracy and overcome the forces of inertia and slip-stick as well as eliminate backlash. The system can also use an encoded area and an opening for connection to an infusion tube with an in-line sensor area where sampling probes can be located to optimized adenosine triphosphate production from the mitochondria in the cells.

### FIELD OF INVENTION

This invention relates to medical systems, and more specifically, to a medical infusion system of any type which provides precise bolus infusions in timing and amounts necessary to develop whole blood oscillations of free hormones, including insulin, delivered to rise in amounts which maintain glucose control when the patient ingests any carbohydrate load in an amount equal to no less than forty percent of the recommended daily allowance of carbohydrates for the person as computed using the weight of the patient. The infusion must maintain relative normality of blood glucose by delivering sufficient levels of hormones in boluses which result in whole blood oscillations of increase and decreases over fifty percent in differential. This system achieves improved and elevated adenosine triphosphate levels effective in treating a multitude of diseases and conditions, by optimizing metabolic pathways.

### BACKGROUND OF THE INVENTION

The historical means of delivering medicines have been arbitrarily divided into two types of delivery. The first is pharmaceutical delivery of compounds through chemically based systems using various carriers with specific chemical properties to control the uptake of the active chemicals. The chemical properties react in pre-designed responses to the tissues which are proximate and affected by the active, thus providing the modulated delivery of medicines.

The second means of delivering medicines uses various mechanical, absorptive and electro-mechanical systems to modulate the delivery, which unlike chemical delivery systems. The current invention provides a unique bolus delivery system which delivers adjusted amounts of precise levels of hormones, and particularly insulin, corrected to achieve elevated adenosine triphosphate, hence the term Medical Infusion Device Improving Adenosine Triphosphate.

Some medical devices have attempted to adjust the delivery of hormones including insulin in conjunction with determining blood glucose levels by various means, and infuse by Continuous Subcutanelous Insulin Infusion (CSII) such as the Medtronic wearable insulin pump with glucose reading information, as in Starkweather, U.S. Pat. No. 6,694,191. There are other truly “closed loop” systems such as the Miles Biostator device as in Clemens, U.S. Pat. No. 4,055,175. While the approach of these devices may constitute an improvement over devices which have no bio-feedback of the patent's response, they still fail to result in body-wide oscillations of hormones necessary to induce improved adenosine triphosphate levels as does the present invention.

The providing of precise bolus deliveries of hormones intravenously, including insulin boluses that achieve therapeutic rhythms (oscillations) and the adjusting the levels and durations between boluses in order to match a physiologically normal response to plasma glucose increases found after ingestion of a high carbohydrate meal in an amount not less than forty percent (40%) of the total suggested daily calories, which delivery addresses and uses individual physiological responses by the patient, and is tailored to cause the blood glucose to remain within a relatively narrow range of 60 to 300 mg/dl and thereby induce increased adenosine triphosphate from carbohydrates and thereby obtain previously unattainable outcomes through the improved metabolic integrity of the cell.

The causing of additional therapeutic effect with precisely delivered and adjusted boluses tailored to provide oscillations (rhythms) which enhance the metabolic integrity of cells and organs through increased levels of adenosine triphosphate are achieved by this now proven method. The purpose of this invention is to provide medical devices which achieve these therapeutic benefits, and particularly new control regimes for selective infusion profiles.

Prior delivery systems including the above closed loop systems ignore the reality that the nominal homeostasis of hormones actually has ever-changing secretion rates of hormones associated with the serum levels of ever-changing glucose levels of the patient.

It might be assumed that increasing the ability to produce adenosine triphosphate would be counter indicated for some abnormal medical conditions but such is not the case. The patient's then-existing physical condition constantly changes on an hourly or even minute by minute basis, but improved adenosine triphosphate abilities is consistently beneficial in all physical conditions yet measured.

Other delivery systems use standardized averaged bioavailability uptake information on a broad basis of patients to determine the allowable dosing of medicines and hormones based chiefly on the patient's weight, and then adjust the treatment amounts, if at all, depending upon the patient outcomes after administration. However these systems do not attempt to achieve gradient changes found in hormone homeostasis which is actually dynamic. These conventional systems are unable to respond to the increase or decrease of available glucose and receptor activities that can change in mere minutes, and thus do not result in increased adenosine triphosphate production by the mitochondria of the cells.

Examples of physiological rhythms which current systems ignore, include almost all endocrine functions from the more obvious circadian rhythms including varying insulin secretion rates, to the very subtle rhythms related to hormonal uptake activities.

The present invention is a means of inducing increased adenosine triphosphate production by the mitochondria within cells by introducing a measured amount of high carbohydrate foods approximating normal requirements for the treatment period, optimally 5 to 6 hours, and by the infusion device providing boluses of hormones and covering oscillations matching the whole blood oscillations necessary to induce improved adenosine triphosphate production. Ongoing real time biological information on circulating glucose is maintained and the hormone infusion adjusted to keep the glucose within the defined level of 60 ml/dl to 300 ml/dl. The biologically superior rhythmic bolus delivery provides momentary changes much like a normal secretion rate, which gradient induces increased adenosine triphosphate levels, a physiological response that is not available with conventional, subcutaneous and thus more gradual, sustained or unchanging rates of hormone delivery.

These momentary changes are the province of this invention which adjusts the hormone delivery in both duration between boluses, and the amount of hormone contained in the bolus, causing differently spaced and differently peaked oscillations of free levels of the hormone causing a dynamic type of hormone and glucose homeostasis, and achieving superior levels of adenosine triphosphates within the cells of the patient.

These oscillations and resulting adenosine triphosphate levels induce normal responses by the cells and cause general corporate-wide benefits including therapeutic effects in the presence of disease states or metabolic deficiencies which are almost immediately ameliorated. Tissue responses to improved adenosine triphosphate are inherently remedial. It is the change in the value of available “free” hormones, not the total amount of free hormones in the patient's system which are needed to become dynamically homeostatic and thus nominal.

The cells, with increased adenosine triphosphate are more able to resist disease, avoid accelerated apoptosis and heal themselves. This is true of all normal cellular activities as the role of DNA is to provide an absolute directive to the cell of its nominal functions, and when a cell is provided nominal or additional adenosine triphosphate through its own mitochondria, the activities of that cell are necessarily and invariably improved.

In the field of pumping, there have long existed numerous ways to pump and infuse liquids for medial use, beginning with a simple syringe or pipette and progressing to highly sophisticated electromechanical software correcting systems of gates and valves with many moving parts and checking systems. In addition there exists a large number of blood and tissue testing diagnostic technologies which can detect, identify and quantify the presence and levels of various substrates in plasma and whole blood. The present inventor's pump described in Zaias et al, U.S. Pat. No. 6,565,535, is particularly useful.

In the present invention, the providing of oscillations and adjustment of patterns of treatment to cover a pre-established carbohydrate load is unique. The oscillations changed in the infusion level and timing of the delivery, adjusted to cover a normal calorie load achieves improved therapeutic responses. The present invention provides a means to deliver this system of timed and accurate boluses resulting in oscillations within the whole blood to treat a number of different conditions and disease states by providing rhythms that are somewhat like changing secretion rates found in healthy man.

Ongoing determination of the levels of carbohydrates in whole blood allow the amount and duration of hormone infusion to match the ingestion of foods or fluids containing carbohydrates and other substrates, and in the preferred embodiment, that determination is more easily achieved by using the existing intravenous access to sample blood and make that determination by reversing the infusion system.

One example of the present invention is the treatment of metabolic diseases, metabolic syndromes, nervous system dysfunctions, burn victims and healing disorders through inducing greatly improved adenosine triphosphate levels as described above, individually tailored and adjusted in delivery, to cause oscillations of circulating hormone levels such as insulin which result in the expression, repression, activation and inactivation of enzymes and other physiological reactions necessary to achieve improved adenosine triphosphate production within the cells, and thus a result of greater cellular energy and heath. Some of the disease states which the inventor has determined respond to improved adenosine triphosphate levels are heart and cardiovascular disease, central nervous system disorders including all types of neuropathy, Alzheimer's disease, Parkinson's Disease, diabetes, retinopathy, psychological disorders, kidney disease, the treating of wounds from injury or surgery, and chronic fatigue syndrome.

First among these diseases which respond to the invention as demonstrated in humans, are metabolic disorders and diseases which are not merely diseases of improper blood glucose, but rather diseases of improper body-wide active and resting metabolism. By using the invention and oscillations of free hormone (insulin) levels meeting the approximate requirements for the above calories of glucose, the resting metabolism of every person, including a diabetic person can be beneficially shifted from elevated lipid and free fatty acid based, to greater carbohydrate based and normal or even greater than normal adenosine triphosphate levels, among other results. The use of the invention for metabolic diseases addresses the core problem which causes disruption of cellular activities and the multitude of so-called “poor outcomes” associated with improper metabolism, diabetes, Alzheimer's disease, Parkinson's disease and general poor cellular energy levels.

In order to demonstrate that increased adenosine triphosphate production could be achieved by the invention, the Bionica PCA 110 infusion device was modified under the direction of this inventor to provide oscillations with changes in the duration and amount of hormone infused. The resulting device was subsequently used in connection with a range of oral carbohydrates approximating that which is normal for the weight and body mass of the patient. Testing was required to obtain the optimal infusion and duration adjustment matrix for patients of various weights and hormone sensitivities. Achieving the desired increased adenosine triphosphate levels and metabolic changes using this method was produced by a trial and error approach to optimize the oscillations necessary to induce the metabolic response.

Prior systems of all types have produced varied and uncertain levels of temporary physiological changes. For example the Biostater was the first device which achieved constant euglycemia which was, at that time, the only goal and desired state. However, consistent euglycemia which is achieved by a constant match of insulin to glucose is not found in healthy man, does not avoid or remedy the various complications of metabolic diseases, and does not provide adenosine triphosphate. It does not achieve measurable true hormone and fuel homeostasis which is very dynamic and ever changing.

This is also true of the Medtronic approach above, which also does not provide the oscillations necessary to normalize metabolism by providing nominal or increased levels of adenosine triphosphate, but rather achieves a constant euglycemia without the cessation of complications, and with the “poor outcomes” associated with diabetes and other metabolic diseases.

This invention does achieve the ever-changing hormone rates found in a more natural course of physiologic responses to carbohydrates, the fuel which provides a greater amount of adenosine triphosphate than any other fuel. Conventional hormone delivery systems of giving glucose to cover insulin subcutaneously is ineffective in providing normalized adenosine triphosphate. This invention thus provides a basic and significant change from prior therapies where infusions were given in set amounts, and counter-reagents adjusted to keep the patient from excursions outside of customary levels.

In one embodiment of the current invention, the device reverses its infusion to allow for the measurement of substrates from whole blood on a real time basis. This avoids manual monitoring of blood conditions and increases the speed and safety of achieving the desired therapeutic changes.

Measurements controlling the infusion timing and amounts can be either by direct measurement or indirectly by the half-life or absorption of the hormone. But to be effective, the infusion must be able to achieve oscillations of hormones as found in whole blood analysis.

Very accurate infusion delivery is beneficial for the invention due to the need to overcome the diluting effect of the normal movement of blood in veins. In order to deliver the treatment in discrete boluses, and achieve oscillations of the type needed as shown by this invention, a very accurate intravenous infusion must be given, as any volume error directly causes a reduction of the oscillations, as well as results in changes to the gradient slope of each delivery. In the past, improvements in basic pumping systems have centered on the use of electronic controls to compensate for the mechanical limitations of pumping systems which generally use non-rigid materials that introduce variations in delivery. Many of these more sophisticated pumping mechanisms have valves and chambers which disturb the reagents normally used in such devices, sometimes for the sole purpose of having a proprietary design. These are not acceptable systems for the infusions to result in discrete oscillations.

The approach to achieving accuracy in pumping has historically been to slow the delivery so that a more precise metering could take place, which is not desired in this invention. Generally available products only offer pump accuracy specifications of plus-or-minus 2 to 5 percent, over the entire reservoir, not for each bolus, thereby making individual deliveries much less uniform and accurate. The preferred embodiment has achieved both accuracy and the required immediate infusion keeping the bolus together during the infusion.

Hormones are proteins which are relatively easily damaged with any type of gate, valve or force which causes shearing upon the opening and closing of the mechanism used to stop the flow. These proteins have the ability to aggregate and become less effective, thereby giving to the patient a treatment which has changed in its effective concentrations. Shear and aggregation can also occur with flows through narrow high pressures. The current invention avoids these problems of shear, and degrading pressures by having no gate or valve, and by having no pressurized closing areas in the travel of the fluid.

Some hormones are delivered in a relatively inaccurate concentration, due to the forces of ionization and collection of medicines on the surfaces of the bag or container being used as a reservoir to store and deliver the medicine. The medicine can collect on the sides of the container, and only delivered in a relatively unknown and short period of time. The current invention avoids this problem by allowing a very accurate delivery and by avoiding a high level of dilution.

Many pumping devices which use syringes have no ability to overcome the natural slip-stick or chatter associated with the storage of energy in the elastic and pliable surfaces and structures, allowing for the syringe moving face (“Plunger”) to move forward in irregular motions. Hysteresis and the natural tendency of Plungers not to move until a force overcomes the inertia and sticking forces cause deliveries by most syringe pumps to be sporadically subject to differing levels of sticking (sticktion). And yet, because of the need for accuracy the other types of infusion devices are not suited. When these other types of infusion devices overcome this inertia and hysteresis, they tend to overrun and deliver at different speeds. The current invention avoids slip-stick, chatter, overruns and the problem of hysteresis by breaking the seating forces in a lateral motion. This allows for extraordinary accuracy while using common plastics. Accuracy of plus or minus one percent of one microliter over a ten milliliter, achieved without error correcting software or other volumetric measurement and control systems which might damage the hormones being infused.

The current invention has only one moving part in relation to the delivery mechanisms. Simplicity allows for more accuracy and lower costs. It also allows for a single handed adoption of the Cassette to the Pumping Device, freeing the other hand and avoiding accidental sticking with “sharps” such as needles which are contaminated with blood or other materials.

Because the preferred embodiment devices avoid slip-stick, chatter and the forces of hysteresis, and still has no gates or valves to damage proteins, it is designed to also be used in a bi-directional application. One of the preferred embodiments herein is the reversal of the cartridge allowing for a precise amount of blood to be withdrawn and available in the closed system to be tested. The preferred embodiment uses this bi-directional accuracy to allow the device to be fitted with any number of probes in line which provide access to measure the properties of a sample, such as blood, and then re-infuse back to the zero point, or if desired, through a second flow direction, and deposit that tested blood into a separate container or depository.

The pumping mechanism can be actuated by any mechanical system which rotably moves the plunger or housing, depending upon the configuration. FIGS. 1, 2 and 3 show a number of different ways to provide this two-axis motion in relationship to the Plunger and Housing. The current invention allows for direct drive, stepper motor, or even spring motor to deliver the amount desired. The “motor” can be even a coordinated hand-eye movement or movement to a series of “click” points.

The reservoir in the preferred embodiment can be pre-filled, thereby enabling the seller to standardize pre-filled reagent cartridges. Also, expensive residues of unused hormones (left-overs) are not necessarily discarded. And there is no need for filling from a container or handling of an additional bottle as pre-filling allows for no filling or common source waste. The cassette can be removed and re-inserted to store the unused reagent for an appropriate period of time in the Cassette.

The design of the motor and assembly allows the pump to be put above, at, or below the heart level, with no resulting change in the delivery profile. This allows the pump to be worn or enclosed in several different tamper-proof or patient access limiting configurations.

The preferred embodiment cartridge when engaged in the delivery device, locks by the rotational providing threads and this locking of the meshed threads makes an accidental infusion by dropping or pressing on the plunger virtually impossible. The cartridge will not siphon out of the pump, or accidentally deliver fluid when dropped or pushed against.

Since the cartridge can also be the pumping system, each time the cartridge is used, it is replaced, and the entire wearing aspects of the pumping system are replaced, thereby causing the product life cycles to be much greater. The entire fluid handling system is replaced with each use, and sterilizing and cleaning of parts is eliminated.

All of the foregoing attributes of the invention and preferred embodiment are beneficial to the proper inducing improved adenosine triphosphate production, and the providing of the preferred embodiment includes all of the above attributes.

### DESCRIPTION OF THE INVENTION

The pumping and aspirating device as seen in FIG. 1, is an embodiment of the invention delivering the required hormone pulses resulting in oscillations which provide the necessary dynamic relationship between rising glucose and oscillations of hormones in the whole blood of the patient. The cassette device with a plunger, a cylinder area where the reagent is filled, a neck opening in the plunger for the connection of the cartridge to a tube which travels to where the infusion takes place, and the in-line area where probes for sampling can be located to provide input to the pumping device, are additional aspects of the invention which help to provide improvements over the basic unique delivery modality.

The Housing can either turn or be affixed to the “Pumping Device” with gearing to link the plunger, cassette, or housing to the motor. The motor can be either electromechanical or a manual wind up, spring or band action motor, adjusted by a mechanical timer for the delivery profile. The motor can also be the manual turning by a hand.

The pumping device can have one, two, three or more sources of input, beginning with the input system to drive the device (“Input”) and a possible input of sensors for in-line for measurement of substrates (“In-line Sensor Probes”) an in-line occlusion pressure sensing system from the line pressure, or an occlusion sensor from back pressure on the motor, (“Occlusion”) and/or input from the reading of the encoded area. Other traditional pump features are intended to be incorporated into the final pumping device system.

FIG. 2 shows the Cassette with only one opening, where the plunger also provides the locking connection system to the infusion tube. A standard lure lock attachment, or other quick connection system would be included in the plunger as a single piece. A “Protective Cap” is shown removed from the single piece cassette. Splines or grooves on the side of the cassette mesh with a gearing mechanism driven by the Motor, all of which are attached to the “Pumping Device” system. The motor rotates the cassette by attachment to splines on the side, with the housing fixed to the pumping device. The plunger then rotates in reference to the cassette, due to the locking of the plunger to the housing by the stanchion.

FIG. 3 shows a direct screwing interface to the side of the cassette to accomplish the rotational and axial movement required to provide the delivery profile, as well as a clamshell opening system for easy removal of the cassette and an internal stanchion to hold the plunger which automatically causes the plunger to turn in reference to the cassette as the motor advances the cassette upwards and downwards in order to infuse and aspirate.

In the preferred embodiment, the rotational velocity exceeds the axial velocity, although with sufficient diameter the difference in rotation travel to axial travel could be adjusted for the flow characteristics of the fluid to be infused and aspirated.

Housings except as to one version of the rack as shown in FIG. 3, can be made in a clamshell or disassembled manner for easy withdrawal.

The delivery profile is designed to allow increasing or decreasing free levels of hormones by infusion in boluses in accordance with the clearing of the hormone by the patient's body.

The purpose of the preferred embodiment is to provide a system of individual boluses of differently spaced and with different amounts of infusion, to respond to changes in the body, such as the amount of free glucose in whole blood.

### SUMMARY

Accordingly, the present invention is a medical infusion system which may or may not have an aspiration system, which infusion system delivers hormone infusions of accurate bolus deliveries at relatively high rates of flow which approximate hormone secretion rate changes necessary and sufficient to cover the action of glucose or other carbohydrates in an amount equal to over 40% of the amount of carbohydrates the patient would take as suggested by standard daily values for the weight and body mass of the patient. The preferred embodiment also avoids the slip-stick, chatter, overruns, and the problem of hysteresis by breaking the seating forces between plunger and cassette wall in a lateral motion that does not vary the delivery profile, and overcomes any viscosity forces of the reagent. The system also eliminates the need to dilute to provide additional control for the limitations of accuracy in other systems. Other important characteristics of the preferred embodiment include disposability, inexpensive cost and use by the manufacturer in glass lined or plastic, and for the cassette to act as both the pumping cartridge and the shipping and storage cartridge thus avoiding loss of reagent in the priming of an infusion device. The preferred embodiment also eliminates the need for withdrawing the medicine with a needle and achieves extraordinary accuracy without error correcting software or expensive volumetric measurement and control systems.

The current invention consists of a pump, and in the preferred embodiment a cassette cartridge pumping and aspirating system. The cassette cartridge contains a plunger, a reservoir area where the reagent is filled, a neck opening for the connection of the plunger or cartridge to a tube which travels to where the infusion takes place, and an in-line sensor area where probes for sampling are located. The in-line area probes can be used to provide input to the pumping device. The preferred embodiment has only one moving part in relation to the delivery mechanism. Simplicity allows for more accuracy and lower costs. It also allows for a single handed adaptation of the cassette to the pumping device, freeing the other hand and avoiding accidental sticking with “sharps” such as needles which are contaminated with blood or other materials.

In a preferred embodiment, the cartridge is cylindrical in shape and has an encoded area. The cartridge may be made of glass, plastic steel or ceramics. It is preferable that part of the outer surface of the cartridge be threaded, and part of the outer surface be grooved to accept and mate with a turning gear. The reservoir area is preferably used for containing a reagent and may be pre-filled.

The neck opening, when not contained in the plunger, is preferably located at the bottom surface of the cassette cartridge and sized to connect an infusion tube to the cartridge. Any conventional tube connection device may be used to connect the infusion tube to the cartridge. The cartridge also has a cap and container top which protects the plunger and its opening, and allows the cartridge to act as the storage vessel for the reagent, and thereby avoid additional steps of filling, mixing, measuring or wasting reagent in the handling of the fluid.

In the preferred embodiment, an optical or electromagnetic strip is located within an encoded area on the cartridge. When the cartridge is filled, an optical or electromagnetic strip with information on the contents and uses of the reagent is placed in the encoded area. The encoded area is preferably located on the outer surface of the cartridge in the area that is first inserted the housing. When the cartridge is placed in the device, it is preferable that the rotational action causes the encoded area to be well aligned and easily read with the uniform motion of screwing the cartridge into place. The preferred rotation, pre-determined position of the encoded area, and the ease of programming a unique character to each cartridge allows the reagent to be mistake limiting.

The preferred embodiment system requires the weight of the patient to be entered into the pumping device, thus reducing the incidence of errors. Any conventional method of storing and retrieving data from the encoded area are preferably included in the present system to limit the incidence of errors. It is preferable that the encoded area comes into close proximity with a reading system as the cartridge is loaded or is first used. The reading system may be any commercially available system capable of reading the encoded area. A medical device stores and uses the encoded information in its operations, including a means to limit the profile of the infusion allowed without further intentional override of the profile.

In the preferred embodiment, the housing consists of a cylindrical tube that is sealed at the upper end, made from plastic and opens at the bottom end to allow cartridge removal. The inner surface of the housing is preferably threaded and sized to receive the cartridge. A plunger is preferably connected to the sealed end and is suspended in alignment with the central axis of the cylindrical tube by a stanchion. In the preferred embodiment, there is a plurality of openings cut through the housing to allow for normally trapped air to be exhausted as the plunger either advances or retards. The plurality of openings also creates an inspection window within the housing allowing visual access and access to the optical or electromagnetic strip within the encoded area. A lip at the bottom of the housing provides for a manually removable cover used to protect the cartridge from contamination or damage to the plunger. When the cartridge is engaged in the housing, the cartridge is locked into place by the rotational engagement of the threads. The locking of the meshed threads makes an accidental infusion by dropping or pressing on the plunger virtually impossible. The cartridge will not siphon out of the pump, or accidentally deliver fluid when dropped or pushed against, which overcomes a major disadvantage to conventional cartridges or syringes.

The preferred plunger is a piston-type plunger and is preferably connected by a stanchion to the sealed end of the housing and is aligned with the central axis of the housing. The plunger head is preferable concaved to trap air and allow for bubble removal, and when an opening is provided on the cartridge, the plunger is sized to fit any reservoir opening, so there is very little dead space thus resulting in very little loss of reagent in the final stroke or at the end of treatment.

In the preferred embodiment, a mechanism is used to rotate the plunger within the cassette and thereby infuse the appropriate amount of hormone to achieve increased adenosine triphosphate and related metabolic actions. The mechanism comprises a gear linkage to the motor, a motor and a gear connection to the cartridge which acts as the pumping device. The pumping mechanism may be actuated by any motor which rotably moves either the plunger or housing, with the other of the two fixed. The present invention allows for direct drive, stepper motor, spring or band action motor, or hand articulation to deliver the desired plunger rotation. The “motor” may be even a coordinated hand-eye movement or movement to a series of “click” points. In a preferred embodiment, the plunger rotates in relation to the walls of the cartridge.

In the preferred embodiment, the cartridge, when placed in the housing, causes the piston plunger to move both forward and aft to infuse or aspirate, and at the same time rotate the plunger within the cartridge to break the forces of inertia and slip-stick as well as eliminate backlash. Because the device avoids slip-stick, chatter and the forces of hysteresis, and has no gates or valves, it is designed to also be used in a bi-directional application, such as one of the preferred embodiments herein, where the precise amount being withdrawn may be distributed, equally or in successive steps of precise delivery, or the precise amount withdrawn re-inserted into the patient to the “zero” point.

A sensor area located in the infusion tube contains probes designed to determine the chemical components and levels of desired substrates in the aspirated whole blood. The determination can be by direct measurement of the medicine, or by measurement of the response of the patient to the medicine. The information obtained by the probes relayed to the pumping device and is used to control or limit the infusion profile, including the changing of both the time between pulses and the amount pulsed.

The bi-directional accuracy of the present invention allows the system to withdraw a measured sample and be used with any number of probes. It is preferable that the probes measure the properties of a sample, such as blood, and then allow the present invention to re-infuse that sample back into the patient after it has been tested which is safe since the system is closed to outside contaminants, or if necessary, by second valve or gate, deposit that blood into a separate container or depository.

The present invention also includes a means to control the cartridge. The pumping system preferably has one, two, three or more sources of input. The preferred pumping device includes, but is not limited to, an input system to drive the device, a sensor input for in-line measurement of substrates, an in-line occlusion pressure sensing system and/or input from the reading of the encoded area, and a means for providing increased adenosine triphosphate. The sensor input receives signals from the in-line sensor probes. The in-line occlusion pressure sensing system determines the line pressure or back pressure on the motor. Other traditional pump features are intended to be incorporated into the pumping device. In the preferred embodiment, the rotational velocity exceeds the axial velocity, although with sufficient diameter the difference in rotational travel to axial travel could be adjusted for the flow characteristics of the fluid to be infused and aspirated.

Since the cartridge also acts as the pump, each time the cartridge is used, it and preferably the plunger are replaced, and the entire wearing aspects of the pumping system are replaced. The product life cycles are much greater and since the entire fluid handling system is replaced with each use and sterilization and cleaning of parts is eliminated.

The purpose of the present invention is to provide improved adenosine triphosphate production by providing measured and spaced boluses of hormones, including insulin, which result in oscillations of free levels of hormones in blood approximating the amount needed to cover at least 40% of a standard carbohydrate meal for that patient. The need to have stimulation like the dynamic homeostasis which produces adenosine triphosphate from carbohydrates was deemed by the Inventor to be a valid approach to induce increases in available cellular energy and thus help the cells to achieve the above therapeutic outcomes.

### DESCRIPTION OF THE DRAWINGS

A preferred embodiment of the present invention is further described in connection with the accompanying drawings, in which:

FIG. 1 is a perspective view of a preferred embodiment of the medical infusion and aspiration system.

FIG. 2 is a perspective view of a preferred embodiment of the cartridge.

FIG. 3 is a perspective view of a preferred embodiment of the housing and plunger.

FIG. 4 is a perspective view of another preferred embodiment of the medical infusion and aspiration device.

FIG. 5 is a perspective view of a third preferred embodiment of the medical infusion and aspiration device.

FIG. 6 is a perspective view of a fourth preferred embodiment of the medical infusion and aspiration device.

FIG. 7 is a perspective view of a fifth preferred embodiment of the medical infusion and aspiration device.

FIG. 8 is a perspective view of an embodiment of the medical infusion and aspiration system having two cassettes being driven independently.

FIG. 9 is a perspective view of an embodiment of the medical infusion and aspiration system having two cassettes coupled by mechanical linkage.

FIG. 10 is a graph of illustrative infusion profiles and free levels of hormones when starting with a glucose load. Several profiles are shown in FIGS. 10A, 10B, 10C and 10D.

### DETAILED DESCRIPTION OF THE INVENTION

The present invention is a medical infusion and possible aspiration system capable of accurate bolus delivery at relatively high rates of flow adjusting the time between boluses and amount of each bolus to provide the required infusion stimulation and resulting oscillations. The present invention provides a means to provide changes in timing and amounts of delivery to provide maximum tissue stimulation while automatically avoiding errors in concentration, reagent and hormone type, and avoiding the problems of shear and other hormone degrading pressure problems. The system can also avoid the slip-stick, chatter, overruns, and the problem of hysteresis by breaking the seating forces between the plunger and cartridge wall in a lateral motion that does not vary the delivery profile by the viscosity of the reagent. The system can also avoid any loss of reagent which occurs in the priming of a cartridge and eliminate the tendency of reagents to separate when in a diluted environment. The invention is disposable, can be inexpensive and may be used by the manufacturer in glass lined or plastic, as both the pumping cartridge and the shipping and storage cartridge. In general, the current invention comprises an infusion pump and possible cassette cartridge pumping and aspirating device. The cassette cartridge pumping and aspiration system consists of a cartridge, a housing, a plunger, a reservoir area where the reagent is contained, a neck opening in the plunger or cassette for the connection of the cartridge to a tube which travels to where the infusion takes place, and an in-line area where probes for sampling may be located. The in-line area probes can be used to provide input to a pumping device. The preferred embodiment has only one moving part in relation to the delivery mechanism and this simplicity allows for more accuracy and lower costs. It also allows for a single handed adaptation of the cartridge to the pumping device, freeing the other hand and avoiding accidental sticking with “sharps” such as needles which may be contaminated with blood or other materials.

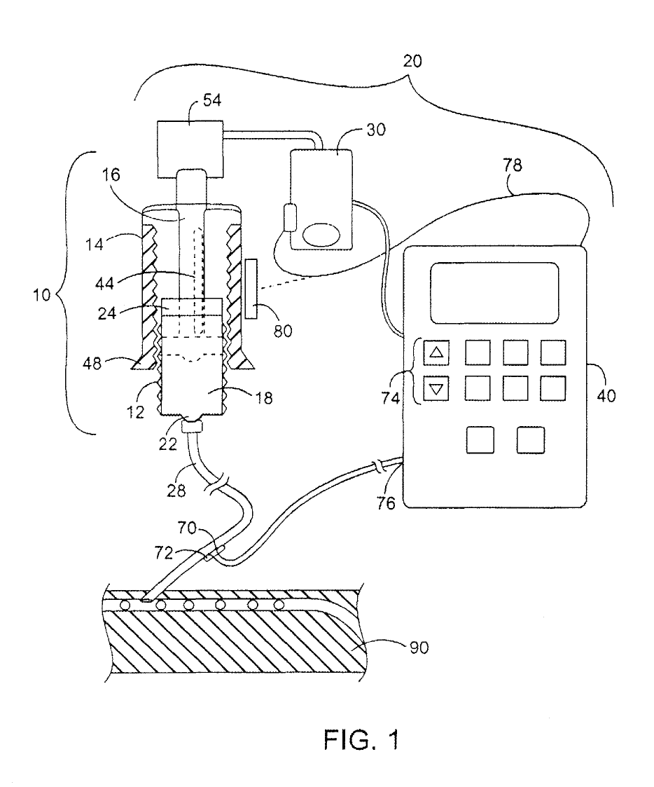

Referring now to the figures, FIG. 1 is a perspective view of an embodiment of the invention showing a cassette 10, a pumping mechanism 20 and a motor 30. The cassette further comprises a cartridge 12, and a housing 14.

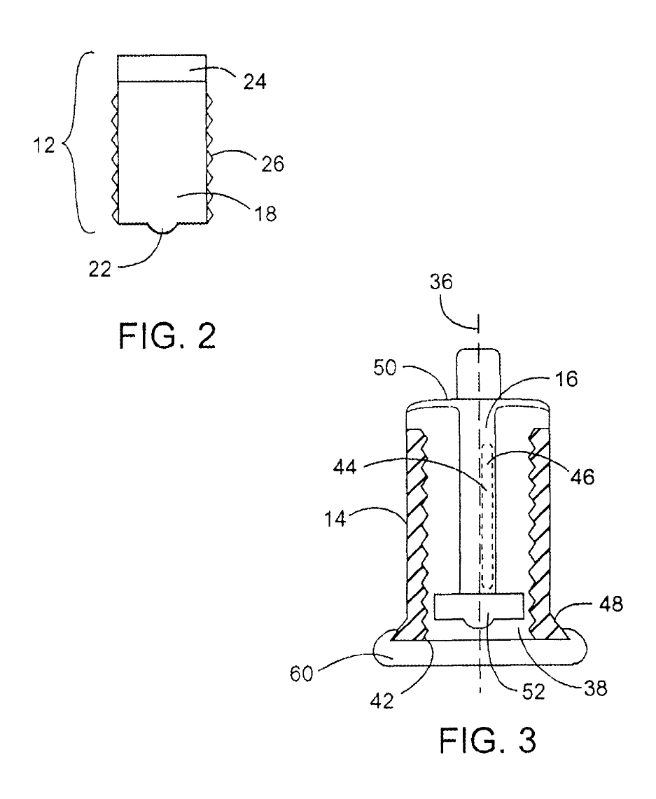

In a preferred embodiment, as best seen in FIGS. 1 and 2, the cartridge 12 is cylindrical in shape and has a reservoir area 18, and encoded area 24. The cartridge 12 is preferably made from glass or plastic. For high-pressure situations, it is preferable that the cartridge 12 be made of steel or ceramics. It is preferable that the outer surface of the cartridge be partially threaded at the top and grooved for the remaining area to the end of the cartridge 26. Any standard or metric thread and groove sizes may be used.

The reservoir area 18 is preferably used for containing a reagent. The reservoir area 18 may be pre-filled, thereby enabling the seller to market pre-filled reagent cartridges which also act as the storage and transportation vehicle. The preferred embodiment eliminates expensive residue that is thrown away with a separate transportation bottle, as pre-filling allows for no waste. The preferred embodiment eliminates dilution requirements due to the accuracy of the pumping means. Cartridges which may be re-inserted can store the unused reagent for an appropriate period of time in the cartridge.

The housing is preferably opened along its center axis to remove cartridges, by means of a hinge 51 which is located off of center to allow the tube to run unobstructed up the stanchion and out of the cassette.

The neck opening 22 is preferably located in the plunger 52, or may be located on the bottom surface of the cartridge 12. The neck is preferably sized to connect an infusion tube 28 to the cartridge 12. Any conventional tube connection device may be used to connect the infusion tube 28 to the cartridge 12. The opposite end of the infusion tube 28 is connected to the sensors and then to a vein in the patient 90.

It is preferable that the cartridge 12 also contains a cap as the container top which allows the cartridge 12 to act as the storage vessel for the reagent, and thereby avoids the additional steps of filling, mixing, diluting, measuring or wasting reagent in the handling of the fluid.

In the preferred embodiment of the invention, an optical or electromagnetic strip is located within an encoded area 24 on the cartridge 12. When the cartridge 12 is filled, an optical or electromagnetic strip with information on the contents and uses of the reagent is placed in the encoded area 24. The encoded area 24 is preferably located on the outer surface of the cartridge 12 in the area that is first inserted the housing 14.

It is preferable that optical reading of a bar code or other reading of the encoded area 24 will minimize dosage mistakes, as each cartridge can set the maximum allowable dose or delivery. When the cartridge 12 is placed in the system, it is preferable that the rotational action causes the encoded area 24 to be well aligned and easily read with the uniform motion of screwing the cartridge 12 into place. The preferred rotation, pre-determined position of the encoded area 24, and the ease of programming a unique character to each cartridge 12 allows the reagent to be mistake limiting. Furthermore, the preferred embodiment system requires a weight to be entered into a pumping device 40 for each patient, and when computed with the allowable dosing based on weight, greatly reduces the incidence of errors. Any conventional method of storing and retrieving data from the encoded area are preferably included in the present system to limit the incidence of errors.

It is preferable that the encoded area 24 comes into close proximity with a reading system as the cartridge 12 is loaded or is first used. The reading system may be any commercially available system capable of reading the encoded area 24. A medical device stores and uses the encoded information in its operations, including a means to limit the profile of the infusion allowed without further intentional override of the profile.

In the preferred embodiment, the housing 14 consists of a cylindrical tube that is sealed at the upper end, as shown in FIG. 3. The housing 14 is preferably made of plastic, however, any suitable commercially available material may be used. The bottom 38 of the housing is preferably open and the inner surface 42 of the housing is threaded. Any standard or metric thread size may be used. A plunger stanchion 16 is preferably connected to the sealed end 50 and is suspended in alignment with the central axis 36 of housing 14. The plunger stanchion is fixed to the housing and is mated with the plunger 52 when the cartridge 12 is inserted into the housing 14. The plunger 52 is fixed to the stanchion and is not allowed to rotate with the cartridge is turned. The housing 14 is sized to threadedly receive the cartridge 12. In the preferred embodiment, there is a plurality of openings 44 cut through the housing 14 parallel to the central axis 36 of housing 14. These openings 44 allow for normally trapped air to be exhausted as the plunger 16 either advances or retards. The plurality of openings 44 also creates an inspection window 46 within the housing 14. The inspection window 46 also allows access to the optical or electromagnetic strip within the encoded area 24. A lip 48 at the bottom 38 of the housing 14 provides for a manually removable protective cap like that used for the cartridge 60 used to protect the housing and the cartridge from contamination or damage to the plunger 16.

When the cartridge 12 the plunger 52 engages the stanchion 16 to lock it into place and then when the cartridge is then engaged in the housing 14, the cartridge 12 is locked into place by the rotational engagement of the threads 26, 42. The locking of the meshed threads makes an accidental infusion by dropping or pressing on the plunger virtually impossible. The cartridge 12 will not siphon out of the pump, or accidentally deliver fluid when dropped or pushed against.

The preferred plunger 52 is a piston-type plunger and is made from plastic, however, any type of non-reactive material may be used. The plunger 52 is preferably connected to the sealed end 50 of the housing 14 and is aligned with the central axis of the housing. The plunger 52 preferably has a concaved face to allow any air to first fill the neck space and be eliminated when the cartridge 12, is inserted into the housing 14, and is preferable sized to fit within the reservoir area 18, so there is very little dead space thus resulting in very little loss of reagent in the final stroke or at the end of treatment.

The plunger 52 and reservoir area 18 configuration may have a larger diameter in relationship to the depth the plunger travels, or a very small diameter and longer plunger travel, depending upon the flow characteristics desired for the application. In very viscous fluids, a different diameter would be helpful for both storage and delivery reasons.

In the preferred embodiment, a pumping mechanism 20 is used to rotate the cartridge with the stanchion 16 and the plunger 52, fixed to the housing 14. Grooves 62 on the side of the cartridge 12 mesh with the gearing mechanism driven by the motor 30, all of which are attached to the pumping device 40. The motor 30 rotates the cartridge 12 by attachment to the grooves 62 on the side of the cartridge, with the housing 14 fixed in relation to the motor or pumping device.

The pumping mechanism 20 comprises a gear linkage 54, a motor 30 and a pumping device 40. The pumping mechanism 20 may be actuated by any motor which rotably moves the stanchion 16 and plunger 52, or rotates the housing 14. The present invention allows for direct drive, stepper motor, spring or band action motor, or hand articulation to deliver the desired plunger rotation. The “motor” may also be a coordinated hand-eye movement or movement to a series of “click” points. In a preferred embodiment, the stanchion 16 and plunger 52 rotate in relation to the walls of the cassette 12.

In one embodiment, a motor 30 with either electromechanical or mechanical operation causes a rotation of the cartridge 12 with the stanchion 16 and plunger 52 fixed to the housing 14, giving both lateral and axial movement of the stanchion 16 and plunger 52. The motor 30 is controlled by an input to cause the pumping and aspiration actions to take place as desired to achieve the free medicine profile. In the case of a mechanical motor, the settings may be made by a spring-like mechanism, with the number of turns and speed of the mechanism being governed by a simple clock mechanism.

The design of the motor 30 and assembly allow the pump mechanism 20 to be put above, at, or below the heart level, with no resulting change in the delivery profile. This allows the pump mechanism 20 to be worn or enclosed in several different tamper-proof or patient access limiting configurations.

The planes formed by the inner surface 42 of the housing and part of the outer surface 26 of the cartridge are positioned so as to allow the cartridge 12 to begin turning as it is first attached, or after the plunger 52 is attached to the stanchion 16. The stanchion 16 may extend beyond the line of the housing 14 for purposes of easy snap-in connection and alignment of the plunger 52 and also the cartridge 12. The number of turns per meter or inch are adjusted to provide the desired rate of flow in both directions. The diameter of the cartridge and its separate housing are adjusted to provide different flow rates and to adjust for any necessary fluid dynamics which might be necessary to pump highly viscous liquids or pump fluids at high flow rates.

As the cartridge 12 is rotationally turned, the device infuses or aspirates liquid, depending on the rotational direction of rotation. The rotational movement of the present invention allows for bi-directional movement and provides accurate infusion or aspiration.

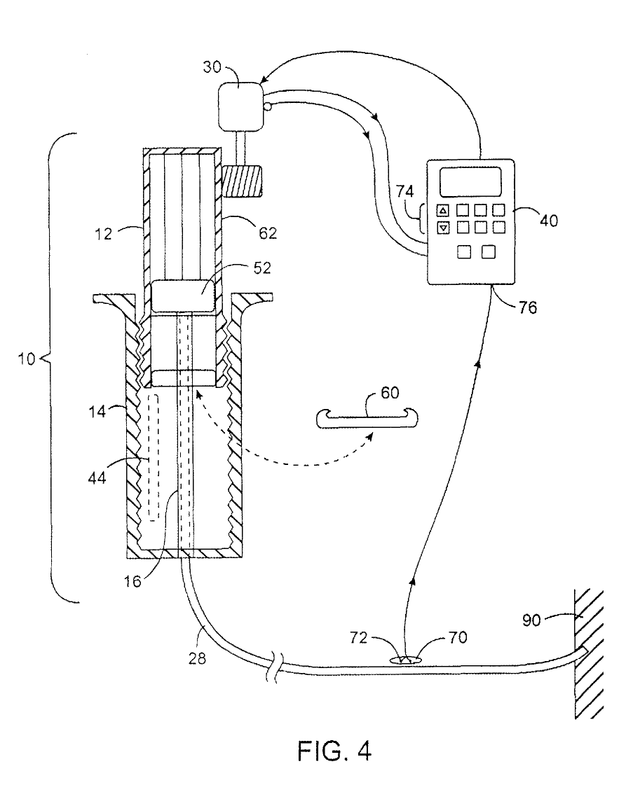

FIG. 4 shows an alternate embodiment of the device. In this embodiment, the cartridge 12 has an opening 22, where the infusion tube 28 is connected to the cartridge. The plunger 16 then rotates, due to the lands and groves meshed between the housing 14 and cartridge 12, causing the infusion and aspiration.

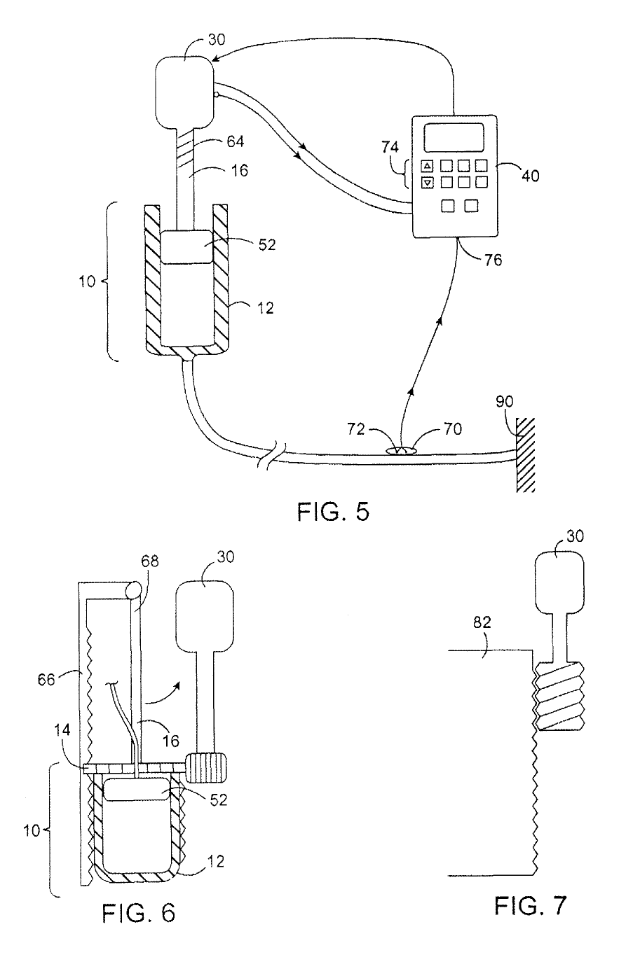

FIG. 5 shows another embodiment of the present invention. In this embodiment, a direct screwing system 64 interface is attached to the side of the cassette 10. The direct screwing system 64 accomplishes the rotational and axial movement of the plunger 52 by dual gearing of the internal rotational drive which automatically causes the plunger 16 to turn as the motor advances the plunger 16 downwards and upwards in order to infuse or aspirate.

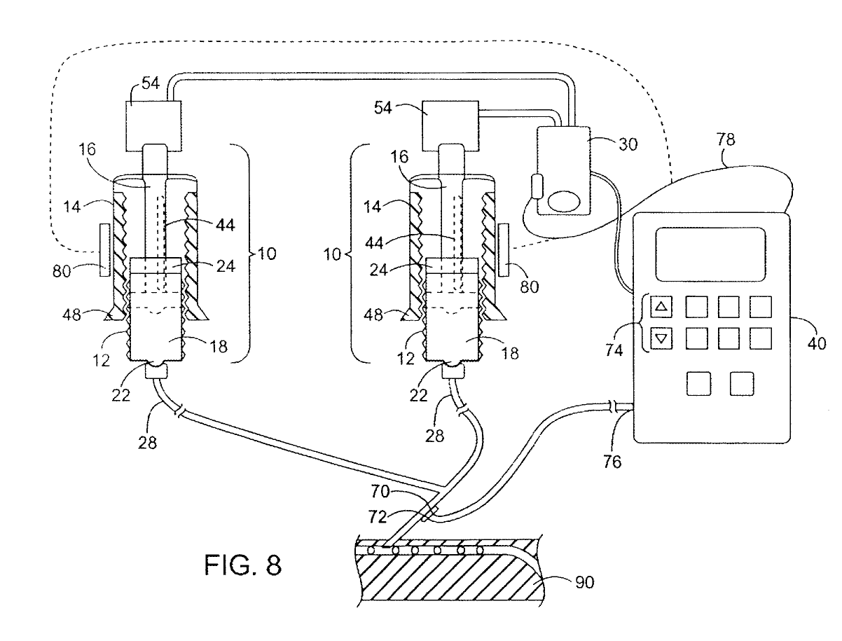

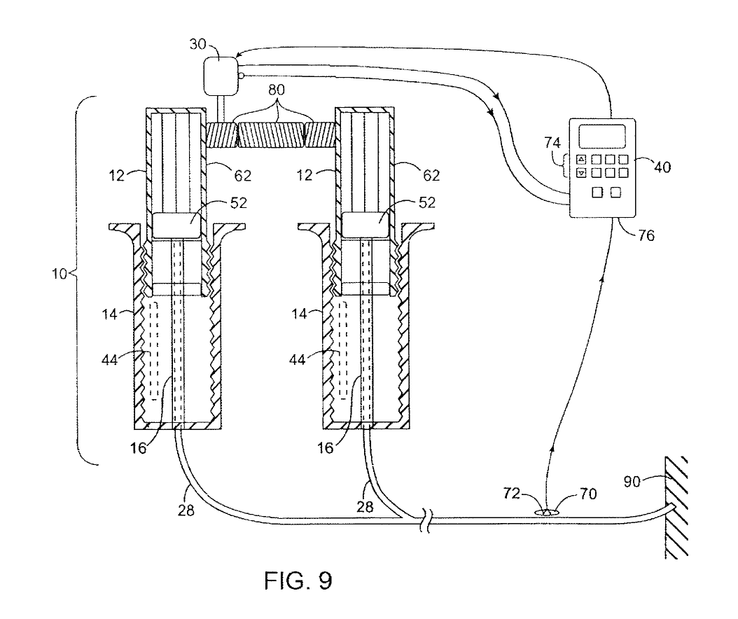

FIG. 6 shows a fourth embodiment of the present invention. FIG. 6 shows a rack 66 threaded surface, which allows the motor 30, when placed adjacent to the rack 66, to turn the housing 14. The plunger 16 remains stationary in relation to the motor 30 and rack 66, thereby causing the plunger 16 to move rotationally in reference to the cassette 10. The plunger sanction 68 may swing away for easy snap-in and snap-out action. FIG. 7 shows a fifth preferred embodiment of the present invention. FIG. 7 shows a side screw 82 configuration for the cassette. FIG. 8 and FIG. 9 show multiple cassettes.

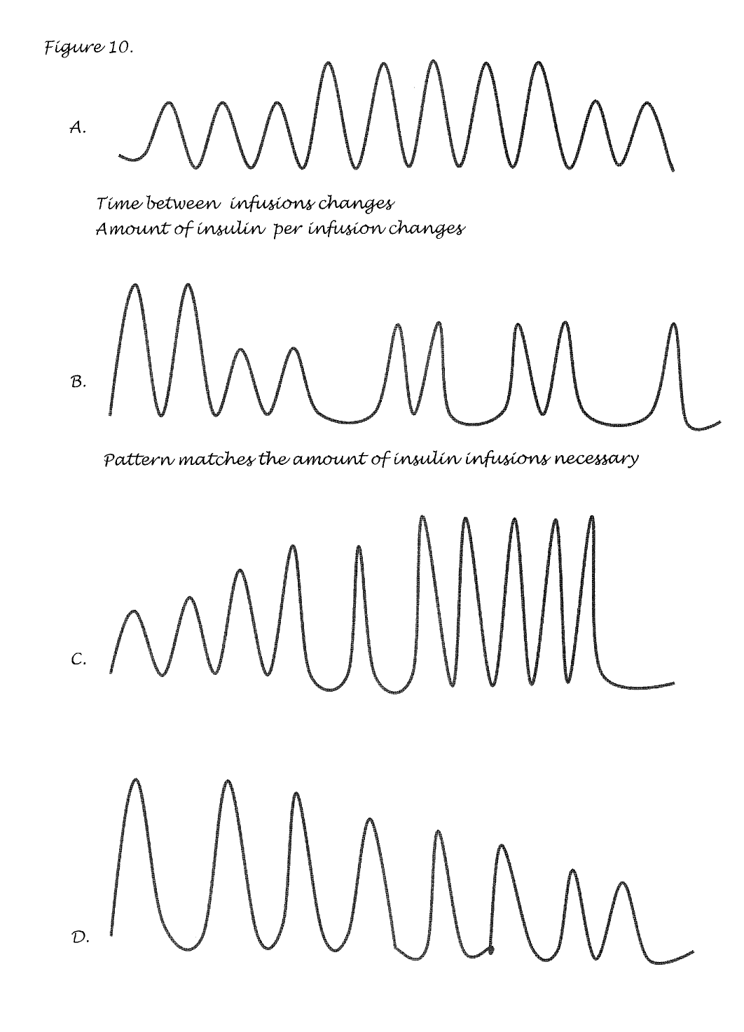

FIG. 10 shows several representative infusion profiles A-D, each where the infusion begins with no or little background level of bioavailable medicine and the time between infusion X is maintained or changed Y with the concurrent amount of each pulse or bolus changed Z. The profile can be changed to provide increased or decreased baselines of free levels of medicines.

In the preferred embodiment, the cartridge 12, when placed in the housing 14, causes the piston plunger 16 to move both forward and aft to aspirate for testing and then infuse, as well as rotate within the cassette 10 to break the forces of inertia and slip-stick as well as eliminate backlash. The infusion is delivered in pulses where the duration between pulses X is changed Y to increase, maintain or decrease the levels of free medicine which causes beneficial responses by tissues to the medicines. Because the device avoids slip-stick, chatter and the forces of hysteresis, and has no gates or valves, it is designed to also be used in a bi-directional application, such as one of the preferred embodiments herein, where the precise amount being withdrawn may be tested and then re-inserted into the patient to the “zero” point.

In the preferred embodiments shown in FIGS. 1, 4, and 5, a sensor area 70 is located within the infusion tube 28. The sensor area 70 contains probes 72 designed to determine the chemical components and levels of desired substrates in the aspirated fluids. The information obtained by the probes 72 relayed to the pumping device 40 and is used to control or limit the infusion profile(s) as contained in FIG. 10. In prototype construction the probes were made of electromagnetic material, however any probe capable of relaying information to the pumping device may be used, including radio frequency, light, infrared waive forms, and chemical testing which is photo sensitive or reactive to the desired substrates.

The bi-directional accuracy of the present invention allows the system to be used with any number of probes. It is preferable that the probes measure the properties of a sample, such as blood, and then allow the present invention to re-infuse that sample back into the patient after it has been tested, or if desired, by second flow direction, deposit that blood into a separate container or depository.

Referring to FIGS. 1, 4 and 5, the present invention also includes a pumping device 40. The pumping device 40 preferably has one, two, three or more sources of input and delivers infusions FIG. 10. The preferred pumping device includes, but is not limited to, an input system to drive the device 74, a sensor input for in-line measurement of substrates 76, an in-line occlusion pressure sensing system 78 and/or input from the reading of the encoded area 80. The sensor input 76 receives signals from the in-line sensor probes 72. The in-line occlusion pressure sensing system 78 determines the line pressure or back pressure on the motor. Other traditional pump features are intended to be incorporated into the pumping device 40.

In the preferred embodiment, the rotational velocity exceeds the axial velocity, although with sufficient diameter the difference in rotational travel to axial travel could be adjusted for the flow characteristics of the fluid to be infused and aspirated.

It is preferable that a second cassette and housing may be coupled and driven either independently or in mechanical linkage 80 with a cassette housing as shown in FIGS. 8 and 9, so as to have as many infusion profiles, either in succession or concurrently as is desired for the given flow profiles and applications, with flows as shown in FIG. 10.

It is a desired effect of the present invention that certain deliveries via long catheters positioned in the patient may benefit from a very stable and accurate system which is not subject to the errors of conventional pumps, even when overcoming higher pressures within a given area.

Since the cartridge and plunger is also the pumping system, each time the cartridge and plunger are used, they are replaced, and the entire wearing aspects of the pumping system are replaced, thereby causing the product life cycles to be much greater. The entire fluid handling system is replaced with each use and sterilization and cleaning of parts is eliminated.

The apparent benefit to having timed boluses of individually controlled amounts, of almost any hormone, as an additional mode for delivery, was deemed by the Inventor to be a valid approach this particular infusion therapy.

The preferred embodiments described herein are illustrative only, and although the examples given include many specifications, they are intended as illustrative of only a few possible embodiments of the invention. Other embodiments and modifications will, no doubt, occur to those skilled in the art. The examples given should only be interpreted as illustrations of some of the preferred embodiments of the invention, and the full scope of the invention should be determined by the appended claims and their legal equivalents.

What is claimed is:

1. A medical infusion system to achieve increased adenosine triphosphates from carbohydrates through the delivery of timed and individually controlled boluses of hormone necessary to produce oscillations in whole blood and sufficient to cover a minimum of a 40% of a normal carbohydrate meal, which boluses are changed by the duration between boluses and amount infused during each bolus irrespective of the existing baseline levels of the hormone, such as insulin, which pulses are delivered by any conventional means of pumping and achieve blood glucose of from 60 mg/dl to 300 mg/dl.

2. The medical infusion device as in claim 1 containing:
a cassette having a cartridge, a housing and a plunger, the cartridge having a cylindrical shape, an outer cartridge surface, and a reservoir area; the outer cartridge surface having a grooved or threaded surface an attachment for an infusion tube,
a pumping mechanism having a gear linkage, a motor and a pumping device delivering a pulsatile infusion,
and,
an attachment for an in-line sensor probe.

2. The medical infusion and aspiration system of claim 2, wherein the outer cartridge surface is in threaded relationship to the housing inner threaded surface.

3. The medical infusion and aspiration system of claim 2, wherein the infusion tube is connectively coupled to the plunger opening.

4. The medical infusion and aspiration system of claim 2, wherein the cartridge is configured to receive a cap and a container top.

5. The medical infusion and aspiration system of claim 2, wherein the housing has no fewer than one and a plurality of openings parallel to the central axis of the housing; the plurality of openings allows for trapped air to be exhausted and creates an inspection window.

6. The medical infusion and aspiration system of claim 2, wherein the bottom surface of the cartridge and housing have lips or ridges to receive a removable cover.

7. The medical infusion and aspiration system of claim 2, wherein the encoded area comprises an optical or electromagnetic strip.

8. The medical infusion and aspiration system of claim 2, further comprising a mechanism capable of reading the encoded area.

9. The medical infusion and aspiration system of claim 2, wherein the stanchion is aligned with the central axis of the housing and configured to capture the plunger fit within the reservoir area.

10. The medical infusion and aspiration system of claim 2, wherein the gear linkage connectively couples the motor to the piston or to the cartridge outer surface.

11. The medical infusion and aspiration system of claim 10, wherein the motor causes both lateral and axial rotation of the plunger.

12. The medical infusion and aspiration system of claim 2, wherein the lateral and axial rotation of the plunger is bi-directional allowing for both infusion and aspiration.

13. The medical infusion and aspiration system of claim 2 wherein the in-line sensor probe is located in the infusion tube.

14. The medical infusion and aspiration system of claim 13, wherein the in-line sensor probe determines a chemical component or level of substrates in an aspirated fluid.

15. The medical infusion and aspiration system of claim 2, wherein the pumping device has at least one source of information input.

16. The medical infusion and aspiration system of claim 2, wherein the information provided adjusts the duration between pulses and amount of fluid pulsed to provide delivery changes to stimulate the production of additional adenosine triphosphate within the cells of the patient.

17. The medical infusion and aspiration system of claim 2, wherein the at least two cassettes are coupled and driven independently or in mechanical linkage.
